(19) United States
(12) Patent Application Publication
(10) Pub. No.: US 2025/0152165 A1
(43) Pub. Date: May 15, 2025

(54) SUTURE PASSING DEVICE FOR ARTHROSCOPIC SURGERY

(71) Applicants: Patrick A. MASSEY, Shreveport, LA (US); Giovanni Francesco SOLITRO, Shreveport, LA (US)

(72) Inventors: Patrick A. MASSEY, Shreveport, LA (US); Giovanni Francesco SOLITRO, Shreveport, LA (US)

(21) Appl. No.: 18/944,130

(22) Filed: Nov. 12, 2024

Related U.S. Application Data
(60) Provisional application No. 63/548,112, filed on Nov. 10, 2023.

(51) Int. Cl. A61B 17/04; A61B 17/06

(52) U.S. Cl. CPC A61B 17/0482; A61B 17/06066; A61B 2017/06085; A61B 2017/061

(57) ABSTRACT

A suture passing device. The suture passing device includes a main shaft having a longitudinal axis, and a tunnel defined therein, the tunnel having a linear portion, a curved portion, and an exit hole at a tip of the shaft. An exit hole axis is disposed at an angle to the longitudinal axis, and the curved portion connects the linear portion and the exit hole. A flexible cannula is passable through the tunnel and the exit hole, the cannula receving a suture therethrough. The exit hole of the tunnel is defined in a side wall of the shaft. An outer sleeve having a bore in communication with an opening disposed at a tip of the outer sleeve is also provided, the main shaft being receivable within the bore of the outer sleeve such that the tip of the main shaft is disposed within the opening of the outer sleeve.

### CROSS-REFERENCE TO RELATED APPLICATIONS

This application claims priority to U.S. Provisional Application 63/548,112, filed Nov. 10, 2023, and entitled “Passing Devices for Sealant Curved Passing of Sutures and Tendons During Arthroscopic Surgery”, the entire contents of which are hereby incorporated by reference.

### BACKGROUND

Knee reconstruction surgery after a ligament or tendon tear often involves drilling transosseous tunnels through bone and passing ligaments, tendon, or meniscus tissue through the drilled tunnel. In current approaches, this is done through an open approach or arthroscopically with the aid of a small camera and the joint being filled with fluid. After drilling a long hole through a tunnel, passing sutures are passed through the tunnel. These passing sutures are then attached to the tissue and used to pull the tissue though the drilled tunnel. Typically, in order to pass the passing sutures, a rigid device is used such as a metal suture passer, to hold the sutures and pass them through the tunnel. These devices are much smaller than the drilled tunnel and allow fluid to pass through the tunnel while passing the suture. Other suture passing devices are rigid and similar to a long cannulated needle. These devices are sharp at the tip which can cause damage of nerves, arteries, and cartilage. There are also some suture passing devices that consist of a flexible stick with the passing suture hanging on the end. These devices are flexible and can get caught in tissue in the joint, creating difficulty of passing. Additionally, such devices only pass in a linear direction and can oftentimes be passed into a dangerous location. Some passing devices have a single curve at the end of the device which can cause significant friction when passing a flexible stick or suture. Current flexible sticks consist of a single cannulated stick with a suture that can be passed through the inner cannulated tunnel. These flexible sticks have only one cannulated tunnel so while one end of the suture is inside of the stick, the other end is outside the stick and can get caught on the bone tunnel. Accordingly, a sealant suture passing device for curved passing of sutures or tendons during arthroscopic surgery that solves the aforementioned problems is desired.

### SUMMARY

According to at least one exemplary embodiment, a suture passing device is disclosed. The suture passing device may include a main shaft having a longitudinal axis, and a tunnel defined in the main shaft, the tunnel having a linear portion, a curved portion, and an exit hole at a tip of the main shaft. An exit hole axis may be disposed at an angle to the longitudinal axis, and the curved portion may connect the linear portion and the exit hole. A flexible cannula may be passable through the tunnel and the exit hole, the flexible cannula receving at least a portion of a suture therethrough. The exit hole of the tunnel may be defined in a side wall of the main shaft. An outer sleeve having a bore in communication with an opening disposed at a tip of the outer sleeve may also be provided, the main shaft being receivable within the bore of the outer sleeve such that the tip of the main shaft is disposed within the opening of the outer sleeve.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

FIG. 1A is a perspective view of an exemplary embodiment of a suture passing device.

FIG. 1B is another perspective view of the exemplary embodiment of a suture passing device.

FIG. 1C is a side view of the exemplary embodiment of a suture passing device.

FIG. 2A is a cross-sectional view of the exemplary embodiment of a suture passing device.

FIG. 2B is a cross-sectional view of the exemplary embodiment of a suture passing device with a flexible cannula and suture therein.

FIG. 2C is a cross-sectional view of the tip of the exemplary embodiment of a suture passing device.

FIG. 3A is a perspective view of the tip of the exemplary embodiment of a suture passing device.

FIG. 3B is another perspective view of the tip of the exemplary embodiment of a suture passing device.

FIG. 4 is a perspective view of the exemplary embodiment of a suture passing device in use.

FIG. 5A is a perspective view of an exemplary embodiment of a flexible cannula of a suture passing device.

FIG. 5B is a perspective view of another exemplary embodiment of a flexible cannula of a suture passing device.

FIG. 6A is a perspective view of another exemplary embodiment of a flexible cannula of a suture passing device.

FIG. 6B is a cross-sectional view of the another exemplary embodiment of a flexible cannula of a suture passing device.

FIG. 7 shows cross-sectional views of exemplary embodiments of flexible cannulas of a suture passing device.

FIG. 8A is a perspective view of an exemplary embodiment of an outer sleeve of a suture passing device.

FIG. 8B is a side view of an exemplary embodiment of an outer sleeve of a suture passing device.

FIG. 9A is a side view of an exemplary embodiment of a suture passing device with an outer sleeve in use.

FIG. 9B is a perspective view of the exemplary embodiment of a suture passing device with an outer sleeve in use.

FIG. 10A is a perspective view of the exemplary embodiment of a suture passing device with an outer sleeve in use.

FIG. 10B is another perspective view of the exemplary embodiment of a suture passing device with an outer sleeve in use.

FIG. 10C is another perspective view of the exemplary embodiment of a suture passing device with an outer sleeve in use.

FIG. 11A is a cross-sectional view of a main shaft and an outer sleeve of an exemplary embodiment of a suture passing device.

FIG. 11B is another cross-sectional view of a main shaft and an outer sleeve of the exemplary embodiment of a suture passing device.

FIG. 12 is a side view of another exemplary embodiment of a suture passing device.

FIG. 13A is a perspective view of another exemplary embodiment of a suture passing device.

FIG. 13B is another perspective view of the exemplary embodiment of a suture passing device.

FIG. 14A is a perspective view of another exemplary embodiment of a suture passing device.

FIG. 14B is another perspective view of the exemplary embodiment of a suture passing device.

### DETAILED DESCRIPTION

Aspects of the invention are disclosed in the following description and related drawings directed to specific embodiments of the invention. Those skilled in the art will recognize that alternate embodiments may be devised without departing from the spirit or the scope of the claims. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

According to at least one exemplary embodiment, a suture passing device for arthroscopic surgery is disclosed. The suture passing device can include a main shaft that is similar in diameter to the drilled hole so the same device that is used to pass suture can also plug the drilled hole, an internal curved channel, so when sutures are passed through it, the sutures pass in a curved manner and exit the suture passing device at an angle with respect to the direction of the drilled tunnel, a conical concentric cannulated plug which can be inserted into the drilled hole to seal the hole from arthroscopic fluid leaving the joint during arthroscopy, and an optional flexible stick that is inserted with the suture to tutor the suture in the inner channel. The suture passing device can further include a tendon passing sleeve with a round tip and an opening that allows to host the extremity of a tendon while is retracted in the drilled tunnel.

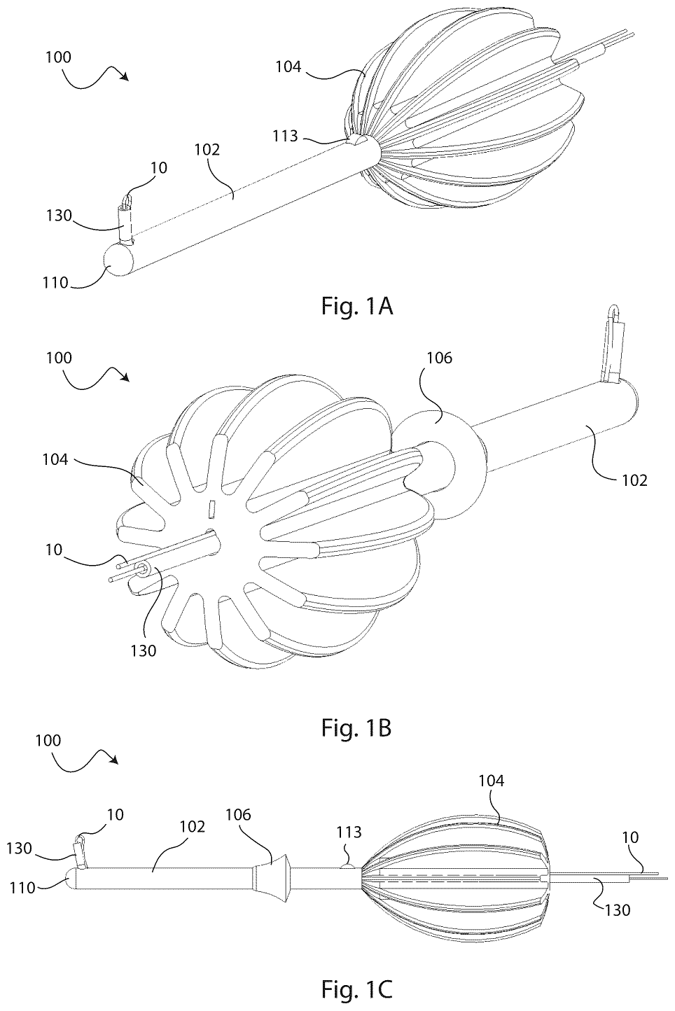

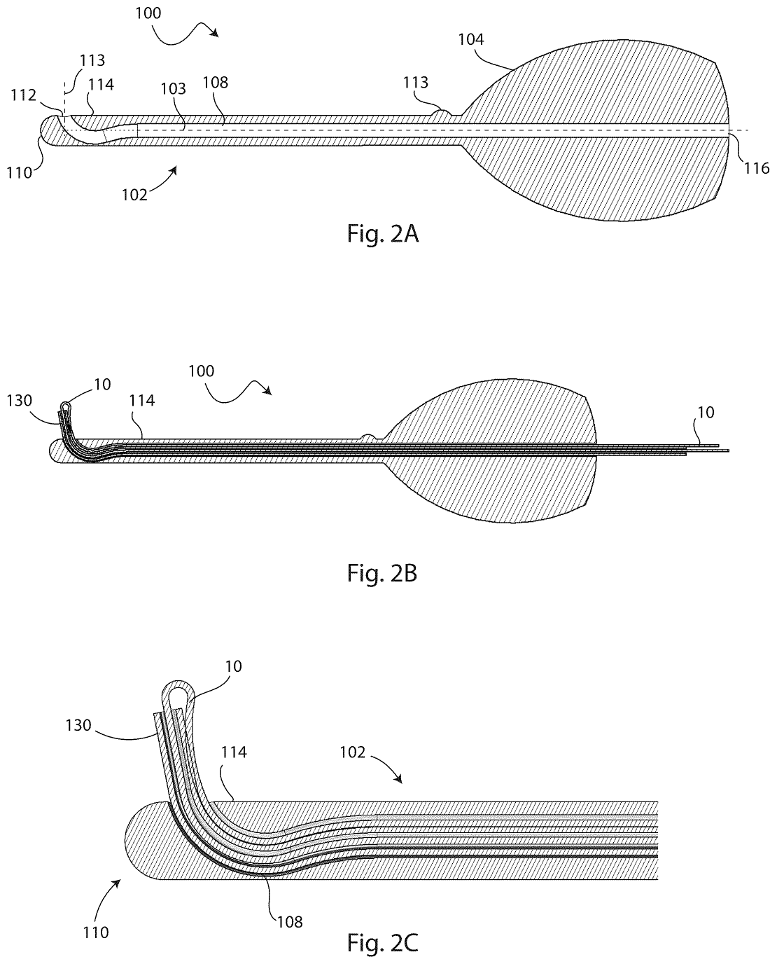

FIGS. 1-2 show an exemplary suture passing device 100. The suture passing device 100 can include a main shaft 102, handle 104, and sealing plug 106. Defined within main shaft 102 and handle 104 may be a cannula tunnel 108. A flexible cannula 130, through which an exemplary suture 10 may be passed, is insertable into cannula tunnel 108.

The tip 110 of shaft 102 may be rounded so as to avoid unintentional penetration of neurovascular structures and are safer to use then sharp suture passing instruments. An exit hole 112 of tunnel 108 is disposed proximate to tip 110 and is defined in a side wall 114 of shaft 102. The exit hole 112 is positioned relative to tip 110 such that the rounded tip pushes native tissue away from the exit hole, and so the passing suture does not impinge on native tissue. Exemplary diameters of shaft 102 may be 7 mm, 8 mm, 9 mm, 10 mm, 11 mm, or 12 mm, while exemplary diameters of tunnel 108 may be 2-3 mm. Exemplary diameters of handle 104 may be 30 mm, 40 mm, 50 mm, or 60 mm. A mark 113 may be provided on the exterior of shaft 302 to indicate the position of the outlet hole. In some exemplary embodiments, the tip may be solid internally (i.e., without any voids therein) and may have a hemispherical or conical shape. In some exemplary embodiments, shaft 102 may have one or more curved portions.

For most of its length, tunnel 108 has a linear portion that is disposed parallel to the longitudinal axis of shaft 102, except proximate to tip 110, where a curve is provided to tunnel 108. The tunnel can be developed in a path that is curved around one or more axes (for example describable with helicoidal or NURBS equations). The cannulated tunnel may further curve in multiple planes to allow for a more gradual curve or larger radius of curvature than possible with a curve in a single plane. In other words, the curved portion of the tunnel may deviate from the longitudinal axis of shaft 102 in more than one direction or plane; as a non-limiting example, the curvature of the tunnel shown in FIG. 2A may be present when seen both from a side cross-sectional view as well as a top cross-sectional view of device 100. The curved path of tunnel 110 can allow the distribution of the suture bending along a longer path and may result in a smaller imposed deflection per unit of length; consequently, the reduced contact stress may result in smaller contact pressure and therefore reduced friction to pass the suture. Exit hole 112 may be defined in side wall 114 of shaft 102, and is thus disposed such that an axis 113 of hole 112 is oriented substantially at an angle of 90° with respect to the longitudinal axis 103 of shaft 102. In other exemplary embodiments, exit hole 112 may be provided such that the axis 113 of hole 112 is disposed substantially at an angle of 45°, 60°, or 75° with respect to the longitudinal axis 103 of shaft 102.

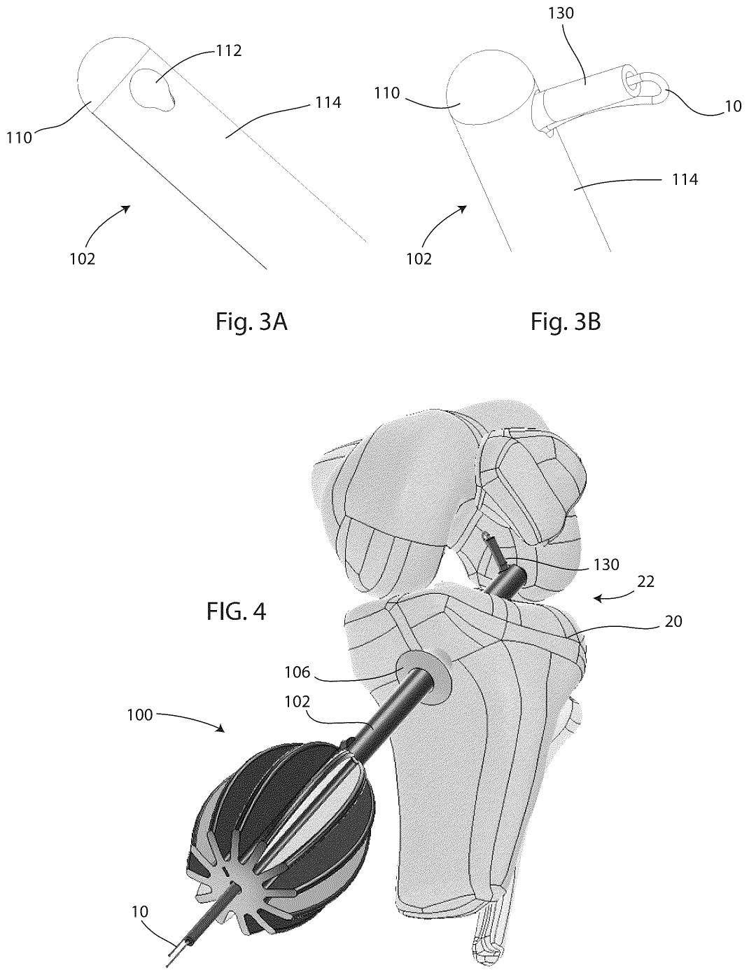

Exit hole 112 may be round, or may have various different shapes adapted to receive flexible cannula 130 and suture 10 therethrough. In an exemplary embodiment as shown in FIG. 3, exit hole 110 may include a notch, or may have the shape of a larger circle and a smaller circle partially overlapping the larger circle. Other shapes for exit hole 112 may be contemplated and provided as desired, so as to cooperate with various embodiments of the flexible cannula, as discussed further below.

FIG. 4 shows an exemplary embodiment of suture passing device 100 in use. The main shaft 102 may be inserted into a tunnel drilled into a bone 20 proximate a joint 22. Flexible cannula 130 with suture 10 may be inserted into an entrance hole 116 of tunnel 108. Sealing plug 106 may be slid on shaft 102 so as to seal an aperture of the tunnel drilled into bone 20, so as to prevent arthroscopic fluid from leaving joint 22 during the arthroscopic procedure.

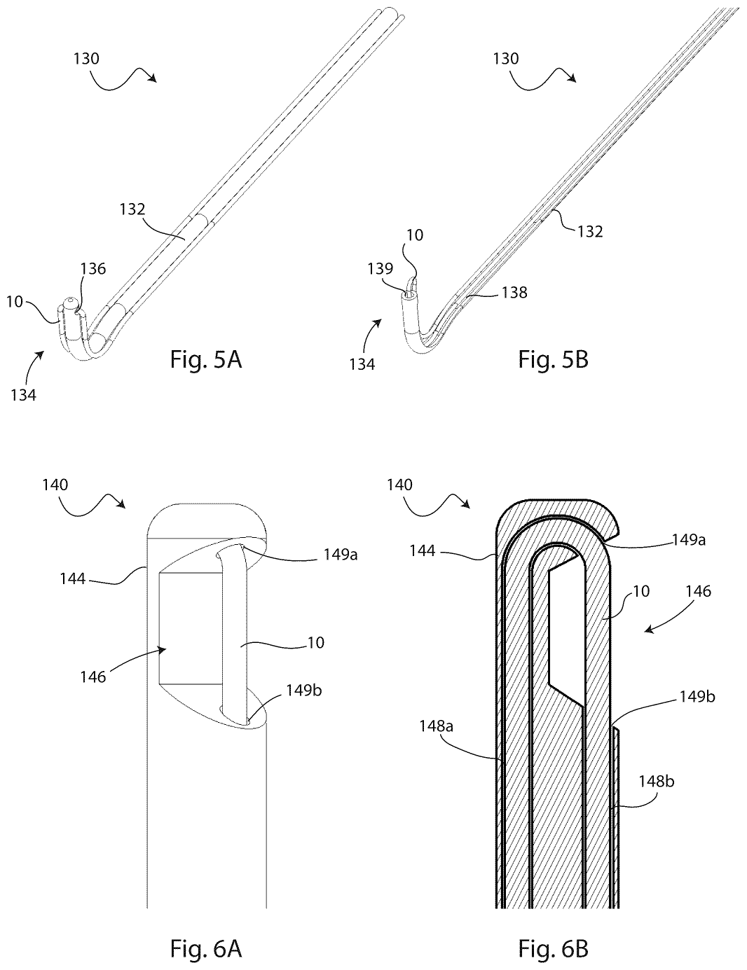

FIG. 5 shows exemplary embodiments of flexible cannula 130. A suture 10 may be pre-loaded into the flexible cannula, or may be loaded by a user as desired. In the embodiment of FIG. 5A, cannula 130 may include a shaft 132 (flexible, but shown with a bend therein) with a distal end 134. A transverse bore 136 disposed through distal end may receive suture 10 therethrough, with both tails of the suture passing externally alongside shaft 132. In the embodiment of FIG. 5B, cannula 130 may likewise include a shaft 132 (flexible, but shown with a bend therein) with a distal end 134. A longitudinal slit or bore 138 may be defined in shaft 132, and have one of the tails of the suture be received therein. At distal end 134, suture 10 may exit the longitudinal slit or bore via a hole 139 defined in the distal end 134. The other tail of suture 10 may then loop back and pass externally alongside shaft 132. In FIG. 5B, shaft 132 is shown as having a semicircular cross-section with a longitudinal slit defined therein. However, it should also be appreciated that shaft 132 may have a circular cross-section and therefore have a longitudinal bore defined therein. In some exemplary embodiments, cannula 130 may be formed from a plurality of materials, thereby allowing the flexibility of the cannula to be tuned or varied along its length.

FIG. 6 shows another exemplary embodiment of a flexible cannula 140. Flexible cannula 140 may be adapted to to hold both tails of the suture such that the suture does not get caught or impinge on any tissue. To that end, flexible cannula 140 may include a pair of bores or slits 148a, 148b defined within shaft 142 thereof. Additionally, a distal end 144 of flexible cannula 140 may be configured to maintain a loop in suture 10 so as to make the suture easier to grab. A notch 146 may therefore be provided at distal end 144, with the notch being in communication with bores or slits 148a, 148b via holes 149a, 149b, respectively. The portion of suture 10 disposed within the notch can therefore be externally accessible.

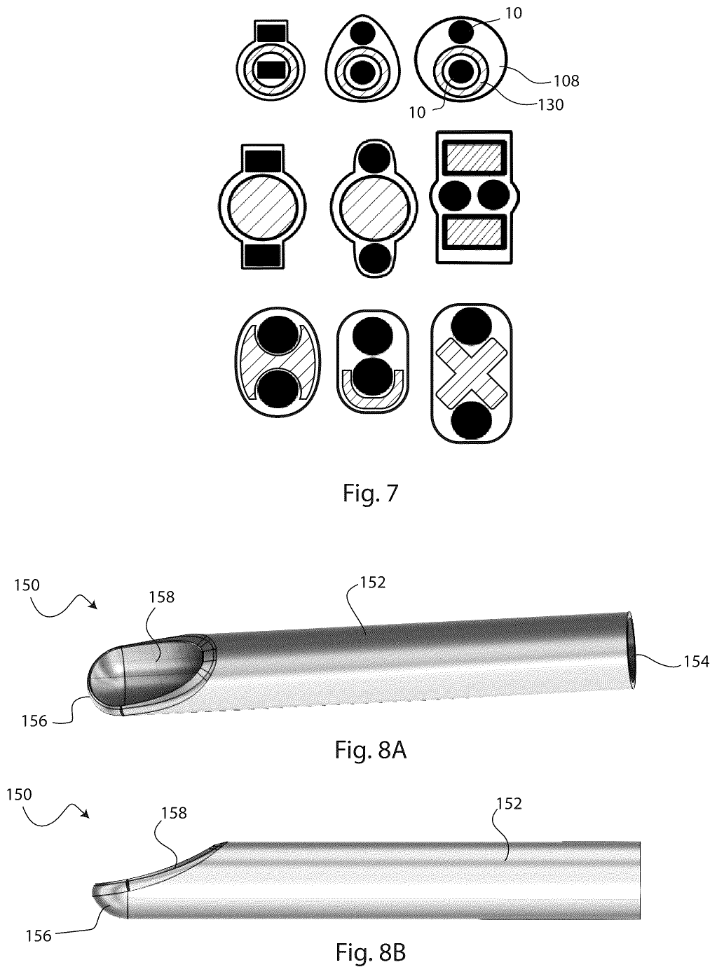

FIG. 7 shows cross-sectional views of various exemplary embodiments of shaft 102 and flexible cannula 130 disposed therein, with the cross-section being taken along a plane perpendicular to the longitudinal axis of shaft 102. The internal wall of tunnel 108 of shaft 102 is represented by the outer enclosing element, the cannula 130 is represented by the diagonally-hatched element, and the suture 10 is represented by the solid black elements. Device 100 may be adapted for sutures with different cross-sections, for example, circular, oval, square, or rectangular cross-sections. In various embodiments, as shown in FIG. 7, flexible cannula 130 may be hollow and cylindrical to host one branch of the suture, solid cylindrical with suture passing externally to the cannula, solid cylindrical or square with sutures disposed internally, H-shaped to partially host the sutures, U- or X-shaped, as shown in FIG. 5. Tunnel 108 may also have various cross-sections that can be shaped to retain the suture and the cannula within so as to improve tutoring of the suture and cannula along the length of the tunnel.

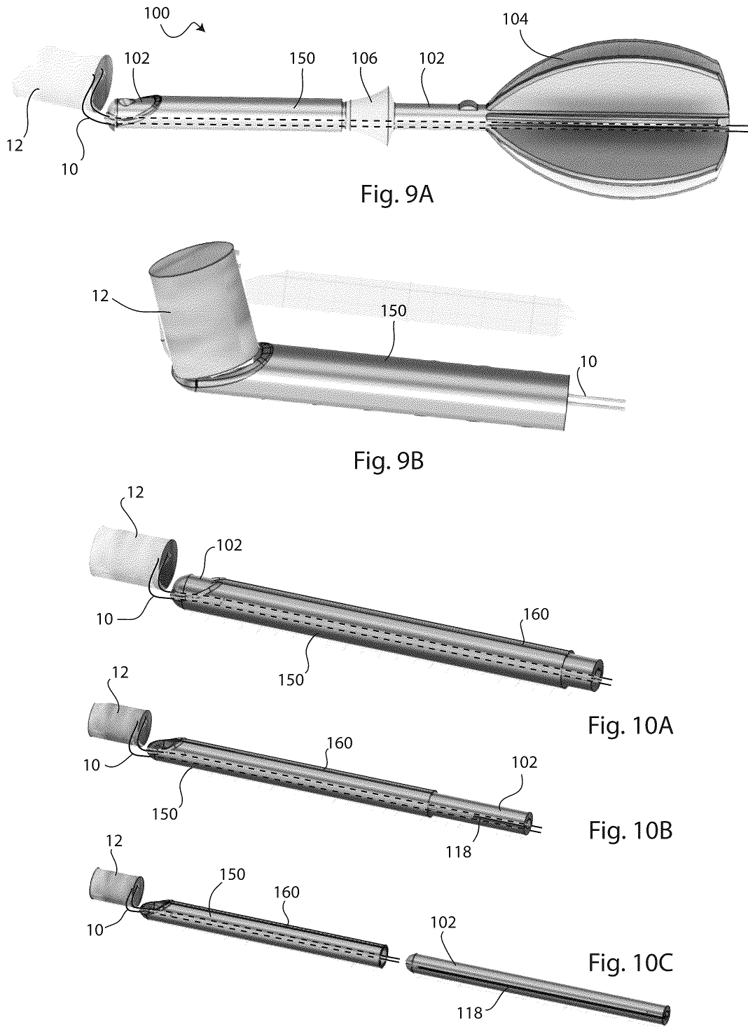

Device 100 may also include an outer sleeve 150 that may be superimposed over shaft 102. Outer sleeve 150 may be used to pass a tendon after the suture has been passed. As shown in FIG. 8, an exemplary embodiment of outer sleeve 150 can include a shaft 152 enclosing a bore 154. A tip 156 of sleeve 150 can include an opening 158, which may have an elliptical shape with curved side edges. Furthermore, the oblong opening 158 may be rounded such that no sharp lips or edges are provided, thereby preventing the opening from damaging any surrounding tissue when being inserted; furthermore, the shape of opening 158 allows for accommodation of an end of a tendon or ligament or other tissue that is larger than the inner diameter of bore 154. Inserting shaft 102 into the bore 154 of sleeve 150 can also block opening 158, thereby increasing the safety of passing sleeve 150 through tissue. Exemplary diameters for outer sleeve 150 may be 7 mm, 8 mm, 9 mm, 10 mm, 11 mm, or 12 mm. The inner diameters of bore 154 may be slightly greater than the outer diameters of a corresponding shaft 102. FIG. 9A shows an exemplary embodiment of device 100 having shaft 102 inserted into outer sleeve 150, with suture 10. FIG. 9B shows tendon 12 passed into opening 158 of outer sleeve 150.

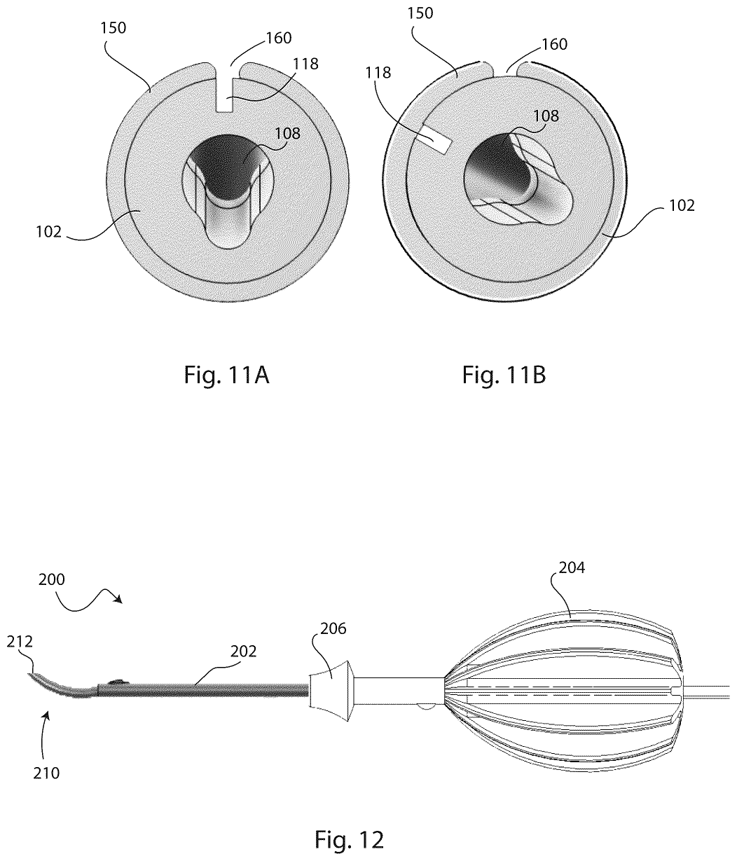

In another exemplary embodiment of device 100, as shown in FIGS. 10-11, shaft 102 may have a longitudinal slit 118 defined therein and extending from the external surface of shaft 102 through to tunnel 108. Outer sleeve 150 may likewise include a longitudinal slit 160 defined therein and extending partially therethrough towards bore 154. Slits 118, 160 can facilitate fast loading of a passing suture into device 100. This can be performed by aligning both slits via rotating shaft 102. Once slits 118, 160 are aligned, a suture 10 may be loaded through the slit. The shaft 102 can then be rotated again, thereby loading the suture 10 into outer sleeve 150.

FIG. 12 shows another exemplary embodiment of suture passing device 200. The suture passing device 200 can include a hollow shaft 202, handle 204, and sealing plug 206. A suture may be inserted directly into hollow shaft 202. Hollow shaft shaft 202 can have an outer diameter of 4 mm and an inner diameter of 3 mm. For most of its length, hollow shaft 202 may have a linear portion that is disposed parallel to the longitudinal axis of shaft 202, except proximate to tip 210, where a curve is provided to hollow shaft 202. The hollow shaft can be developed in a path that is curved around one or more axes (for example describable with helicoidal or NURBS equations). The hollow shaft may further curve in multiple planes to allow for a more gradual curve or larger radius of curvature than possible with a curve in a single plane. The curved path of hollow shaft 202 can allow the distribution of the suture bending along a longer path and may result in a smaller imposed deflection per unit of length; consequently, the reduced contact stress may result in smaller contact pressure and therefore reduced friction to pass the suture. An exit hole may be provided at tip 210 of shaft 202, and is disposed such that an axis of hole 212 is oriented substantially at an angle of 90° with respect to the longitudinal axis of shaft 202.

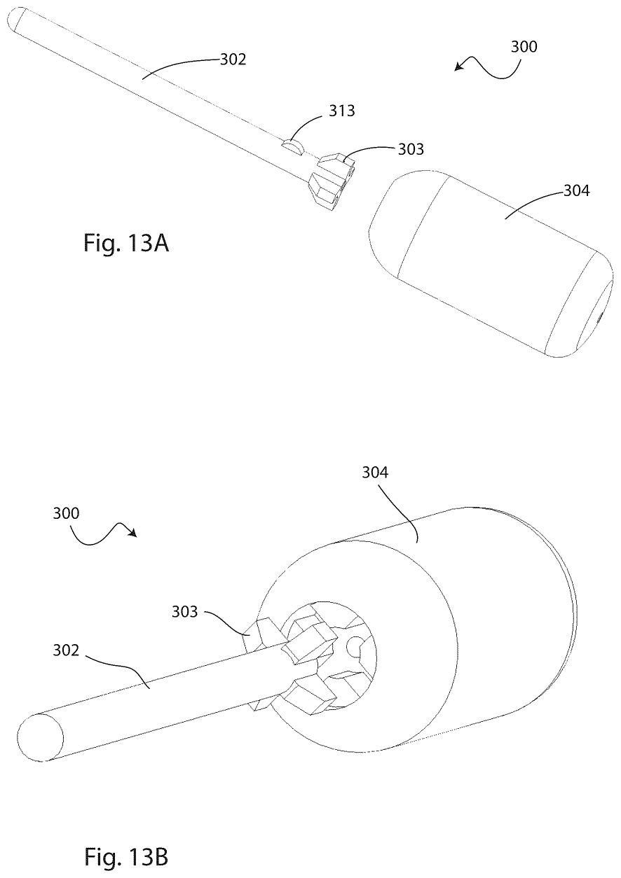

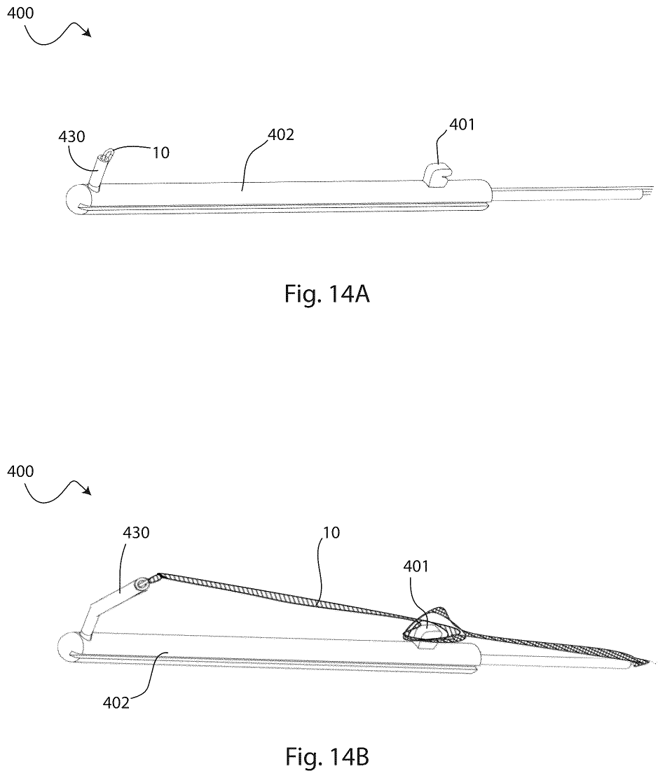

According to another exemplary embodiment of suture passing device 300, main shaft 302 may be separable from the handle, rather than being integrated with the handle. Additionally, main shaft 302 may be provided with connectors 303 to plug it into a multi-use handle 305, as shown in FIGS. 13A-13B. A mark 313 may be provided on the exterior of shaft 302 to indicate the position of the outlet hole.

According to another exemplary embodiment of suture passing device 400, main shaft 402 may have suture retaining structure 401, which may be provided as a orientation piece with a v-shaped slit, as shown in FIG. 14A. Sutures 10 from flexible cannula 430 can be locked on structure 401, for example by positioning the suture in the slit. When advancing the cannula 430, the locked sutures 10 may tether cannula 430, such that it flexes or bends down as shown in FIG. 14B.

The foregoing description and accompanying figures illustrate the principles, preferred embodiments and modesn of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A suture passing device, comprising:
a main shaft having a longitudinal axis; and
a tunnel defined in the main shaft, the tunnel having a linear portion, a curved portion, and an exit hole at a tip of the main shaft;
wherein an exit hole axis is disposed at an angle to the longitudinal axis; and
wherein the curved portion connects the linear portion and the exit hole.

2. The suture passing device of claim 1, further comprising a flexible cannula passable through the tunnel and the exit hole, the flexible cannula receving at least a portion of a suture therethrough.

3. The suture passing device of claim 2, further comprising at least one bore or slit extending through the flexible cannula, wherein the at one bore or slit at least partially surrounds a portion of the suture.

4. The suture passing device of claim 2, further comprising:
two bores extending through the flexible cannula; and
a notch defined proximate a tip of the flexible cannula, the bores opening into the notch;
wherein the suture is receivable within the two bores; and
wherein, when the suture is disposed within the two bores, the suture is externally accessible via the notch.

5. The suture passing device of claim 1, wherein the exit hole is defined in a side wall of the main shaft.

6. The suture passing device of claim 5, wherein the exit hole axis is perpendicular to the longitudinal axis of the main shaft.

7. The suture passing device of claim 5, wherein the exit hole axis is disposed at an angle of 45°, 60°, or 75° with respect to the longitudinal axis of the main shaft.

8. The suture passing device of claim 1, wherein the tip of the main shaft is rounded.

9. The suture passing device of claim 1, further comprising a handle disposed at an end of the main shaft that is opposite the tip.

10. The suture passing device of claim 1, further comprising a sealing plug slidably disposed on the main shaft.

11. The suture passing device of claim 1, further comprising an outer sleeve having a bore in communication with an opening disposed at a tip of the outer sleeve, wherein the main shaft is receivable within the bore of the outer sleeve such that the tip of the main shaft is disposed within the opening of the outer sleeve.

12. The suture passing device of claim 11, further comprising a first longitudinal slit defined in the main shaft and a second longitudinal slit defined in the outer sleeve.

13. The suture passing device of claim 1, further comprising a suture retaining structure disposed on the shaft.

14. The suture passing device of claim 1, wherein the tip of the shaft is solid and has a hemispherical or conical shape.

15. The suture passing device of claim 1, wherein the cannula is formed from a plurality of materials.
